(19) United States
(12) Patent Application Publication
(10) Pub. No.: US 2026/0049988 A1
(43) Pub. Date: Feb. 19, 2026

(54) METHOD OF BREAST CANCER BIOMARKER DETECTION USING A BIOSENSOR BASED ON A FIBER-OPTIC BALL RESONATOR

(71) Applicant: Autonomous Organization of Education "Nazarbayev University", Astana (KZ)

(72) Inventors: Kanagat KANTOREYEVA, Astana (KZ); Aliya BEKMURZAYEVA, Astana (KZ); Madina SHAYMERDENOVA, Astana (KZ); Zhuldyz MYRKHIYEVA, Astana (KZ); Zhannat ASHIKBAYEVA, Astana (KZ); Daniele TOSI, Astana (KZ); Takhmina AYUPOVA, Astana (KZ)

(21) Appl. No.: 19/284,969

(22) Filed: Jul. 30, 2025

(30) Foreign Application Priority Data
Aug. 14, 2024 (KZ) 2024/0977.2

(51) Int. Cl. G01N 33/574; G01N 21/47; G01N 33/543; G01N 33/68

(52) U.S. Cl. CPC G01N 33/57415; G01N 21/47; G01N 33/54386; G01N 33/6872; G01N 2021/4709; G01N 2201/08; G01N 2333/70585; G01N 2333/765

(57) ABSTRACT

Disclosed is a method for detecting breast cancer biomarkers, which can be applied in cancer diagnosis and monitoring the effectiveness of cancer therapy. This fiber-optic biosensor-based approach employs advanced photonic techniques to identify specific biomarkers with high sensitivity and specificity. The technical result is achieved through a system including a fabricated and biologically functionalized fiber-optic biosensor, immersed in a controlled fluidic environment that mimics in situ conditions. The biosensor is integrated into a commercially available catheter, which is placed within a flow-through tube containing serum. The work focuses on enhancing biosensor performance under simulated blood-like conditions, including optimization of sensor positioning, packaging modifications, and assessment of specificity and sensitivity under varying pressure levels. The aim is to improve the efficiency, accuracy, and reliability of cancer biomarker detection, ultimately enabling earlier and more precise diagnosis for patient benefit.

### CROSS-REFERENCE TO RELATED APPLICATIONS

Not applicable.

### FIELD OF THE INVENTION

The proposed method for breast cancer biomarker detection can be applied in cancer diagnosis and in monitoring the response to cancer therapy. This fiber-optic biosensor-based approach leverages advanced photonic technologies to detect specific cancer biomarkers with high sensitivity and specificity. By integrating principles of both biochemistry and optical engineering, the method lies at the intersection of biomedical diagnostics and bioengineering.

CD44, a biomarker linked to cancer, is crucial in understanding the progression and metastasis of the disease. Therefore, accurately detecting and measuring CD44 is significant for clinical diagnosis of cancer. Considering this, creating platforms for detecting CD44 is prioritized, particularly using biosensors that are both sensitive and selective. This developed packaged biosensor can identify the CD44 cancer biomarker in a dynamic environment, aimed towards clinical use.

### BACKGROUND OF THE INVENTION

Available assays for detecting CD44 protein, a breast cancer biomarker, include the solid phase ligation assay (research only), the commercially available OncAlert™ point-of-care device for oral cancer diagnosis, several commercially available ELISA (enzyme-linked immunosorbent assay) kits, and several studies on biosensors (not commercialized). OncAlert™ test reads out results in 15 minutes, however it only provides a qualitative estimate and not quantitative results. ELISA provides quantitative results, but it requires sample preparation and is not highly sensitive. When comparing currently available biosensors against CD44 protein developed worldwide compared to the biosensor built by our group, the main strengths of existing biosensors (electrochemical and photoelectrochemical) lie in their high sensitivities, being tested with cells.

However, these transducers are harder to use in vivo due to electrical interference. An optical fiber biosensor is a good alternative for an easy and cheap diagnostic, as it does not necessitate electrical connections and is not affected by electric interference like electrochemical biosensors (Mowbray and Amiri 2019). Other inherent advantages include biocompatibility, small size, compactness, lightness, resistance to electromagnetic interference, and low cost of production (Marazuela and Moreno-Bondi 2002; Mehrvar et al. 2000). Our platform while having all these advantages also offers ease of fabrication, low attomolar level detection capabilities and detecting analytes in a wide concentration range.

### SUMMARY OF THE INVENTION

The primary purpose of our method is to address and overcome the significant limitations of traditional cancer diagnostic methods, such as delayed detection, and limited sensitivity and specificity. Early detection is critical for improving patient outcomes, as it allows for timely intervention and treatment. Our method for detecting breast cancer biomarker uses an ultra-sensitive fiber-optic ball resonator. This method of implementing a fabricated sensor works in a blood-mimicking environment and maintains its accuracy even under pressure changes.

### BRIEF DESCRIPTION OF DRAWINGS

FIG. 1 shows the step-by-step fabrication of the sensor of the present invention.

FIG. 2 shows the functionalization of the sensor for the fabrication of the biosensor.

The manufactured system with the packaged biosensor is shown in FIG. 3.

### DETAILED DESCRIPTION OF THE INVENTION

The technical result is achieved by a system for detecting breast cancer biomarker, which consists of a fabricated and biologically functionalized biosensor (1) specially immersed in an environment close to in situ conditions. An in vitro detection system was developed to mimic the blood circulation in veins for measuring CD44 protein levels. The system used a syringe pump (2), Legato 100 KD Scientific, that had the capability to provide adjustable flow rates. The pump's flow rate was set at a constant value of 20 mL/min for imitating blood flow in veins. The CD44 protein, with concentrations ranging from 7.1 aM(attomolar) to 16.7 nM(nanomolar), was passed through a thin tube system (3) with a diameter of 1 mL at the specified flow rate. Before performing protein measurements, the internal part of the experimental tubing was blocked to reduce non-specific binding by treating it with a 1% bovine serum albumin (BSA) solution. The measurement of the CD44 protein was performed using a diluted calf serum at the ratio of 1:10. CD44 protein with concentrations of 16.7 nM,12.9 pM, 9.3 fM, 7.1 aM and a total volume of around 60 mL each were prepared for dynamic measurement. The specificity tests were done with two control proteins: thrombin and gamma globulin. The measurement setup consisted of a cannula (4) in the range from 14-gauge to 20-gauge (G) made of polyurethane (PUR). To ensure the integrity of the biofunctionalized sensor during measurements while maintaining its exposure to the target analyte, a small rectangular section of the upper part of the catheter was removed and the tip was sealed to safeguard the integrated fiber-optic ball resonator. This is reflected in the upper left part of FIG. 3. The length of this cannula can be chosen from a range 20-100 mm, and its interior diameter was 0.5-2 mm. The biosensor (1) was placed within the tube (3) using a tricot bevel needle (5) to create a hole. The sensor was connected to the LUNA OBR(Optical Backscatter Reflectometer) 4600 device (6) (Luna Inc., Roanoke, VA, USA). A schematic illustration of this in vitro system is shown in FIG. 3. Holder (7) was used to keep the cannula in place and stable. Waste glass (8) was used to collect the waste solution flowing through the tube.

This proof-of-concept work used a commercially available catheter (cannula and tube system) to integrate the sensor that was then put inside the tube with a flowing serum. The work aimed to further enhance the performance of the biosensor in the blood-mimicking system with more in-depth experiments of the in vitro setup. It includes additional study of the following: optimization of placement of the sensor inside the tube/catheter; modification of the packaging of the sensor in blood-mimicking environment; and specificity tests with control proteins and assessments of pressure insensitivity.

With respect to prior works, the packaging hereby designed accomplishes a three-fold objective: allows the fiber positioning in line with respect to the vessel along which the detection takes place; prevents the measurement artefact due to the presence of the outer walls mimicking a blood vessel; prevents the measurement artefact due to the packaging itself acting over the ball resonator. The configurations used in prior studies were designed for static tests, whereas this catheter is optimal for dynamic measurement since it prevents the artefacts due to the relative motions of the ball resonator with respect to the blood vessel and to the catheter itself. Since the ball resonator biosensor (1) is a sensor with very low reflectance, the placement of sensors in this way allows for obtaining a clean measurement even during the motions due to the pumping system (2).

The sensor's packaging and placement were optimized to create a better environment for the fabricated ball resonator's performance in blood-mimicking environment; Calibration of the fabricated fiber-optic ball resonator sensors in both static and dynamic conditions showed similar sensitivity to the refractive index change demonstrating its usefulness as a biosensing platform for dynamic measurements; The fabricated sensors were shown to be insensitive to pressure changes further confirming their utility as an in situ sensor; Incubating increasing protein concentrations with antibody-functionalized sensor resulted in nearly instantaneous signal change indicating a femtomolar detection limit in a dynamic range from 7.1 aM to 16.7 nM; The consistency of the obtained signal change was confirmed by repeatability studies; Specificity experiments conducted under dynamic conditions demonstrated that the biosensors are highly selective to the targeted protein; Surface morphology studies by AFM measurements further confirm the biosensor's exceptional sensitivity by revealing a considerable shift in height but no change in surface roughness after detection.

Tests have shown that the sensor can detect very low protein concentrations (7.1 aM to 16.7 nM) almost instantaneously. The sensor is also highly selective, detecting only the target protein. The studies confirmed the efficiency and sensitivity of this method of using the sensor even under dynamic conditions.

This utility model focuses on the study of CD44 protein detection with an in-house fabricated fiber-optic ball resonator biosensor in vitro (Kaur et al. 2022) by employing dynamic conditions to mimic blood flow circulation in the vein. Previously, the same developed optical fiber biosensor has demonstrated the sensitivity and specificity in measuring CD44 in static conditions with a limit of detection at the attomolar level (Bekmurzayeva et al. 2021; Bekmurzayeva et al. 2022). The protein levels were also detected in an in vitro setup mimicking blood flow, highlighting the potential for this technology in practical diagnostic applications.

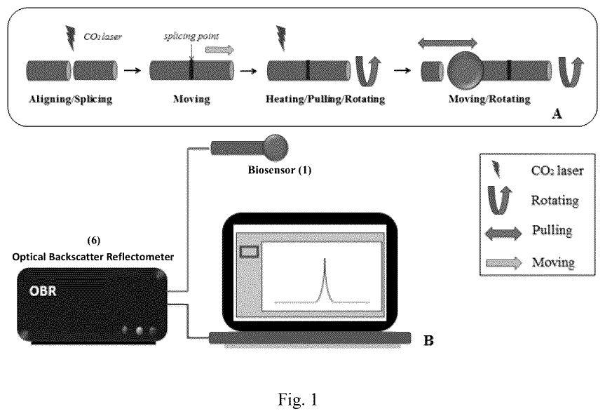

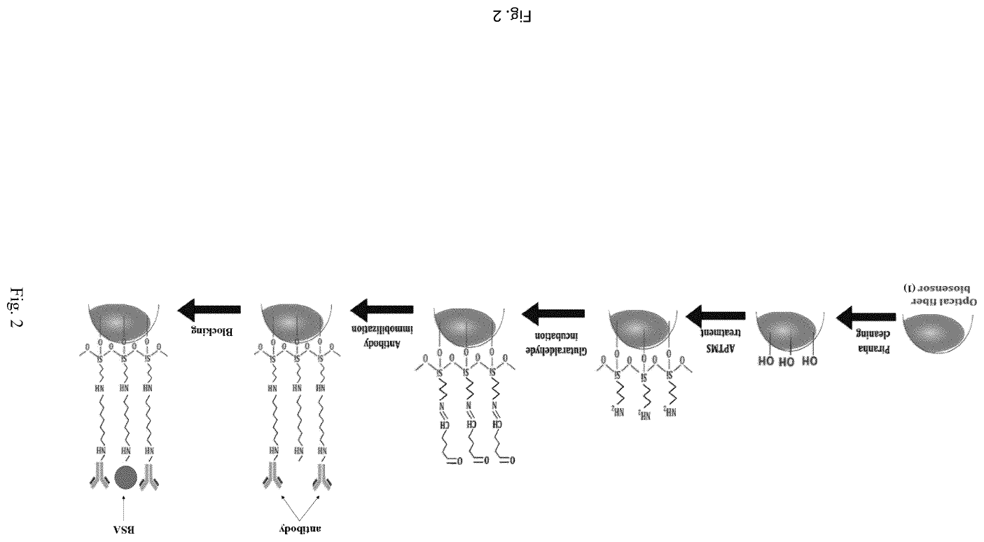

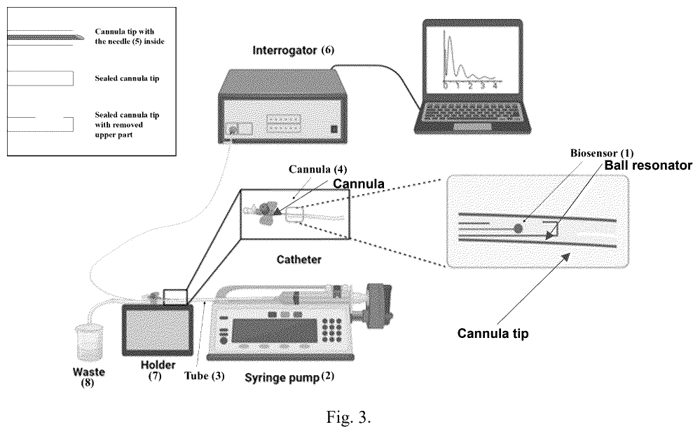

A silica single-mode fiber (SMF), SMF-28e+ (Corning, NY, USA), was utilized to construct an optical fiber ball resonator. The SMF-28 has a core size of 8.2μm and a cladding size of 125μm. The fabrication of an optical fiber ball resonator was completed using a CO2 splicing device Fujikura LZM-100 (Fujikura, Japan). Throughout the fabrication process of the ball resonator sensor, the equipment underwent calibration, and the suitable splicing Mode 9 and ball lensing Mode 43 were chosen. The ball resonator's requirements, including its diameter (around 500μm), were adjusted using the Fiber Processing Software. The Fujikura LZM-100 was used to create a spherical ball lens by heating and spinning the optical fiber. Precise modifications were carefully done to optimize the size and quality of the ball resonator, including adjustments to its power, rotation speed, and feeding speed (Relative power—100; Break add power—130; Rotation—150; Movement—0.2). The instrument was set up, and two prepared SMF-28 were aligned and joined using a CO2 laser. The process involved the stripping, cleaving, and positioning of the fibers within the device. The fabrication procedure involved aligning, splicing, heating, and rotating the fibers, which resulted in the formation of a spherical resonator at the end of the fiber. FIG. 1 illustrates the fabrication process of the optical fiber ball resonator from SMF-28. The length of the fabricated sensor was approximately 12 cm. Then it was stripped and spliced using a splicing machine to attach the pigtail connector. Finally, the ball resonator was spliced and connected to the interrogator (LUNA OBR 4600 (Luna Inc., Roanoke, VA)). FIG. 1 shows the step-by-step process of the fabrication and calibration of the sensor.

Among the patented objects with a similar manufacturing method to this one, there is a Sensing device based on balloon-shaped optical fiber MZI (Mach-Zehnder Interferometer) and manufacturing method of balloon-shaped optical fiber MZI sensor (CN113324570A, 2021). This fabrication method has the advantages of small volume, convenient fabrication, low cost, high reliability, which solves the problem of light leakage in the traditional balloon-shaped optical fiber sensor into the environment from a cladding. Another patented Gas-ball-shaped MZI sensor, manufacturing method thereof and sensing system based on MZI sensor (CN113777345A, 2021) has similar properties. This fabrication method solves the problems of easy optical fiber breakage and low visibility of interference fringes in their prior work.

The most similar patented object with a similar objective is the micro-optical fiber biosensor of markers for breast cancer in a kind of quick detection serum (CN110132896A, 2019). This fiber optic sensor is immersed in a liquid containing breast cancer biomarker and is very sensitive to changes in the environment. However, this sensor uses a flame melting method, which is not a reproducible fabrication method.

The innovative integration of optical fiber biosensor technology in a single, portable device represents a significant advancement in the field of cancer diagnostics, promising to improve patient care and outcomes significantly. The use of optical fiber biosensors for detecting biomarkers such as CD44, with demonstrated sensitivity and specificity in dynamic in vitro conditions, underscores the potential of this device to revolutionize cancer diagnostics.

1. A method for detecting a breast cancer biomarker using a biosensor based on a fiber-optic ball resonator, comprising an ultra-sensitive fiber-optic sensor with a low detection limit, wherein detection is performed in vitro by simulating venous blood circulation for measuring CD44 protein levels, the system comprising a syringe pump configured to provide adjustable flow rates approximating venous blood flow, a tubing system through which CD44 protein in concentrations ranging from attomolar to nanomolar is passed, wherein the tubing is pre-treated with a blocking agent, specifically bovine serum albumin (BSA), to reduce non-specific binding, the biosensor being enclosed in a catheter-like device formed of a flexible polymeric cannula with an inner diameter and length suitable for sensor insertion, and connected to an optical backscatter reflectometer for real-time signal detection, the system being further configured to assess specificity using control proteins and to remain functional under variable pressure conditions.
